(19) United States
(12) Patent Application Publication
(10) Pub. No.: US 2025/0104825 A1
(43) Pub. Date: Mar. 27, 2025

(54) METHOD AND APPARATUS FOR PROCESSING PROTEIN INTERACTION DATA

(71) Applicant: CANON MEDICAL SYSTEMS CORPORATION, Otawara-shi (JP)

(72) Inventors: Russell HUNG, Edinburgh (GB); Simon FISHER, Edinburgh (GB)

(73) Assignee: CANON MEDICAL SYSTEMS CORPORATION, Otawara-shi (JP)

(21) Appl. No.: 18/473,372

(22) Filed: Sep. 25, 2023

(51) Int. Cl. G16H 20/10; G16B 15/30

(52) U.S. Cl. CPC G16H 20/10; G16B 15/30

(57) ABSTRACT

A medical information processing apparatus comprises processing circuitry configured to: receive protein-protein interaction data; estimate, using the protein-protein interaction data, a first set of proteins based on the first drug and a second set of proteins based on the second drug; and determine, based on the first set of proteins and the second set of proteins, if there is at least one protein which is influenced by both the first drug and the second drug.

### FIELD

The present invention relates to systems and methods for processing protein interaction data, in particular when applied to protein-protein interaction networks.

### BACKGROUND

Biological networks are naturally occurring networks that consist of a scale-free topology with nodes of varying node degrees. Biological networks can be used to represent the interactions of molecules, such as proteins, which do not work alone but exert effects on other molecules. A protein-protein interaction network (PPI) represents individual proteins (or their respective protein coding genes) as nodes that are connected by edges to other proteins with which they share some functional significance. Edges between nodes can represent a canonical physical interaction, a direct protein fusion or a correlation in bioavailability (co-expression). The likelihood of a protein having n interactions exponentially decays with n+1. The nodes in a network around a specific protein can be referred to as the local neighbourhood or local interactome of that protein.

Polypharmacy is a rapidly growing domain in healthcare due to an aging and increasingly comorbid population. This is particularly relevant in high income countries (HICs), where individuals accumulate morbidities over a lifetime while having continued access to high quality healthcare. Polypharmacy is loosely defined as the administration of three of more drugs to a patient, with some definitions stating >5 drugs. Typically, polypharmacy is applied to target disparate conditions, but can sometimes be used to treat the same condition in resistant individuals, e.g. when more than two different drug families are used in resistant hypertension or diabetes. Polypharmacy can also be applied in situations where the aetiology of a first disease causes a secondary disease, e.g. when secondary hypertension arises due to renal damage. The treatment of secondary disease may be treated differently to primary disease and can involve polypharmacy.

It is useful for clinicians to understand polypharmacy when evaluating patients who are poor-responders to treatment or who have experienced suspected adverse drug reactions (ADRs). It is also useful for clinicians to consider the effects of polypharmacy when treating highly medicated patients. Moreover, a greater understanding of polypharmacy is important during the pharmaceutical R&D process so that the effect of new drugs on patient biology, including potential biological effects, drug-gene interactions, and contra-indications can be evaluated.

Current attempts to understand polypharmacy focus on predicting new links in knowledge graphs. These analyses involve producing a large knowledge graph representing drugs and their adverse effects. However, investigating the effects of drug combinations based on their known adverse effects ignores the roles of the proteins and genes that mediate these effects. Genes and proteins are fundamental in explaining the occurrence of these adverse effects. Homeostatic feedback mechanisms allow a cell to respond to the perturbation of a drug on a protein (this process, over time, underpins developing drug resistance). Therefore, the local neighbourhood around a drug target can be affected. Overlapping local neighbourhoods in context of polypharmacy can be crucial in underpinning possible resistances or ADRs. An understanding of interactions between local neighbourhoods is also important for interpreting pharmacodynamic and pharmacokinetic responses. There is therefore a need to explore polypharmacy in a way that considers the interactions among these intermediate molecules.

### BRIEF DESCRIPTION OF THE DRAWINGS

Embodiments are now described, by way of non-limiting example, and are illustrated in the following figures, in which:

FIG. 1 is a schematic diagram of an apparatus according to an embodiment;

FIG. 2 is a schematic diagram of a method in accordance with an embodiment;

FIG. 3 is a flow chart illustrating in overview a method in accordance with an embodiment;

FIG. 4 is a graph illustrating the threshold calculation;

FIG. 5 is a displaying image of the local interactome of a drug target in accordance with an embodiment;

FIG. 6 is a displaying image of the local interactomes of three drug targets in accordance with an embodiment; and

FIG. 7 shows the displaying image of FIG. 6 with further annotations.

### DESCRIPTION

Certain embodiments provide a medical information processing apparatus comprising processing circuitry configured to: receive protein-protein interaction data; estimate, using the protein-protein interaction data, a first set of proteins based on the first drug and a second set of proteins based on the second drug; and determine, based on the first set of proteins and the second set of proteins, if there is at least one protein which is influenced by both the first drug and the second drug.

Certain embodiments provide a method comprising: receiving protein-protein interaction data; estimating, using the protein-protein interaction data, a first set of proteins based on the first drug and a second set of proteins based on the second drug; and estimating, based on the first set of proteins and the second set of proteins, if there is at least one protein which is influenced by both the first drug and the second drug.

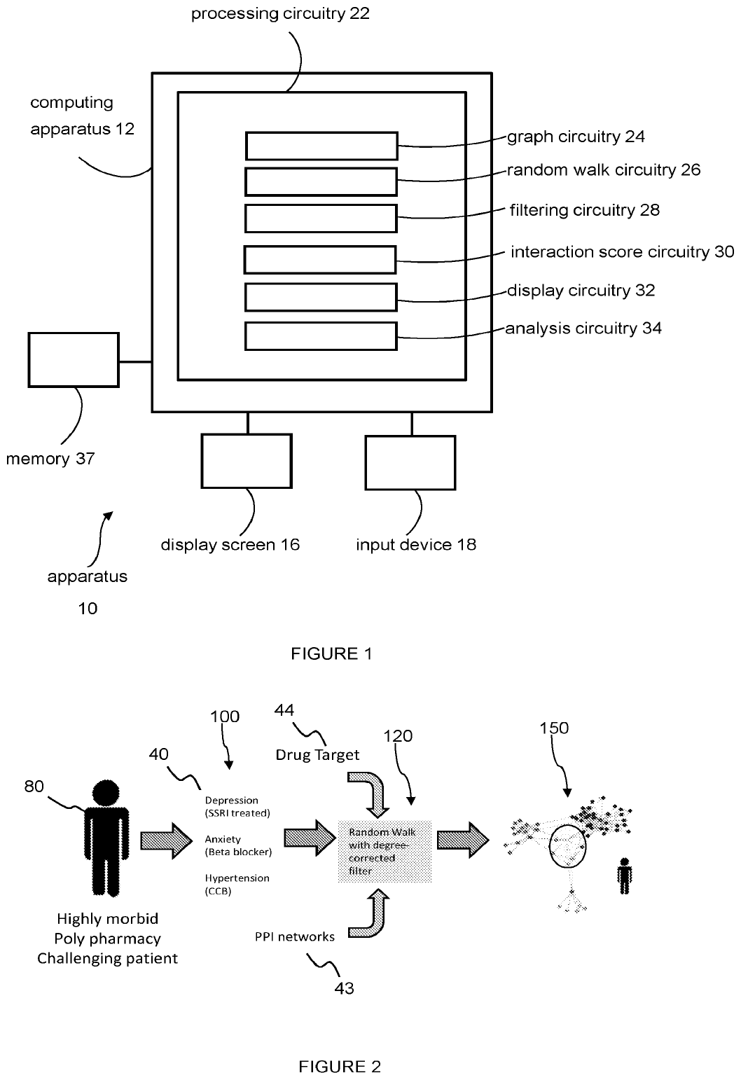

An apparatus 10 according to an embodiment is illustrated schematically in FIG. 1. The apparatus 10 may also be referred to as a medical information processing apparatus. The apparatus 10 is configured to process protein-protein interaction data. The apparatus 10 is further configured to display an image based on the protein-protein interaction data.

The protein-protein interaction data may be any type of data that comprises a set of proteins and a representation of the interactions between the respective proteins. An interaction between a pair of proteins may correspond to a canonical physical interaction, a direct protein fusion or a correlation in bioavailability (co-expression). In addition, an interaction between a pair of proteins may be predicted. A predicted interaction between a pair of proteins may be based on a consideration of the location in the genome of the genes that code for the respective proteins as well as a consideration of the topology of the genome.

Protein-protein interaction data is a type of omics data. Omics data relates to information obtained through high-throughput experiments to characterize and quantify biological molecules. In other embodiments, the apparatus 10 may be configured to process other types of omics data, such as genomics or metabolomics data. In other embodiments, the apparatus 10 may be configured to process any appropriate data that can be represented using a graph.

The apparatus 10 comprises a computing apparatus 12, which in this case is a personal computer (PC) or workstation. The computing apparatus 12 is connected to a display screen 16 or other display device and an input device or devices 18, such as a computer keyboard and mouse. The computing apparatus 22 receives data from memory 37, which may also be referred to as a data store or storage. In alternative embodiments, computing apparatus 12 receives data from one or more further data stores (not shown) instead of or in addition to memory 37. For example, the computing apparatus 12 may receive data from one or more remote data stores (not shown), which may comprise cloud-based storage.

The memory 37 stores protein-protein interaction data. The protein-protein interaction data may be stored in any file format suitable for storing text-based data or for storing graph representations, such as TSV, CSV, XLS, XML. In other embodiments, the protein-protein interaction data may be stored in another suitable memory, for example in another apparatus or in a cloud-based memory.

Computing apparatus 12 comprises a processing circuitry 22 for processing data. The processing circuitry 22 comprises a central processing unit (CPU) and Graphical Processing Unit (GPU). The processing circuitry 22 provides a processing resource for automatically or semi-automatically processing protein-protein interaction data.

The processing circuitry 22 includes a graph circuitry 24 configured to receive the protein-protein interaction data and convert the protein-protein interaction data into a graph representation if the protein-protein interaction data is not already in such a structure; a random walk circuitry 26 configured to perform random walks on a graph based on a graph based on the protein-protein interaction data and output a list of count values for the proteins corresponding to the nodes visited; a filtering circuitry 28 configured to filter the proteins based on the count values; an interaction score circuitry 30 configured to calculate an interaction score for the proteins; a display circuitry 32 configured to display the graph, and an analysis circuitry 34 configured to perform further analysis on the list of proteins.

In the present embodiment, the circuitries 24, 26, 28, 30, 32 and 34 are each implemented in the CPU and/or GPU by means of a computer program having computer-readable instructions that are executable to perform the method of the embodiment. In other embodiments, the circuitries may be implemented as one or more ASICs (application specific integrated circuits) or FPGAs (field programmable gate arrays).

The computing apparatus 12 also includes a hard drive and other components of a PC including RAM, ROM, a data bus, an operating system including various device drivers, and hardware devices including a graphics card. Such components are not shown in FIG. 1 for clarity.

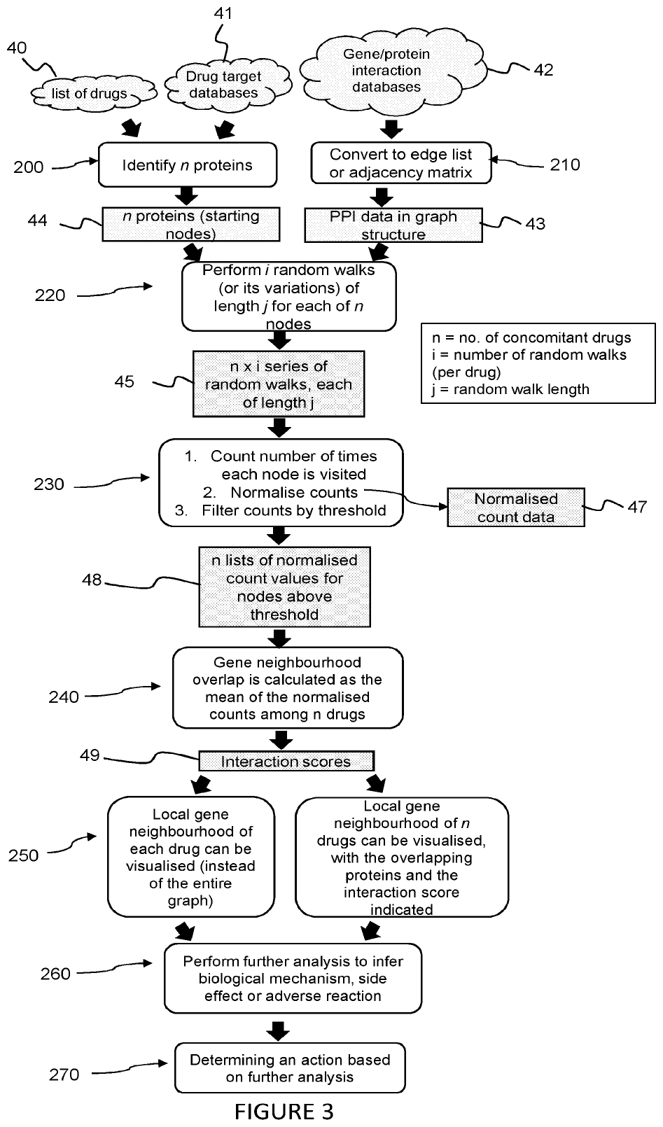

The processing circuitry 22 of FIG. 1 is configured to perform a method in accordance with FIGS. 2 and 3. FIG. 2 is a schematic of a method for identifying proteins influenced by a first drug and a second drug, and FIG. 3 is a flow chart illustrating an overview of this method.

As illustrated in FIG. 2, in one embodiment, at stage 100, a user receives a list of n drugs 40 that are prescribed to a subject 80, where n is greater than or equal to two. In the present embodiment, the drugs are, respectively, for the treatment of depression, anxiety and hypertension. In other embodiments, any other combination of drugs may be chosen. The user then identifies n proteins 44 that are each respectively associated with the n drugs 30. In the present embodiment, each of the n proteins are a drug target of each of the respective drugs and are identified by the user searching a drug target database. Of course, it will be appreciated that in some embodiments, a drug may have a plurality of targets, in which case the respective protein 44 associated with a drug may be one of the plurality targets for that drug.

At stage 120, the graph circuitry 24 receives the n proteins 44 and a graph data structure 43 comprising a protein-protein interaction (PPI) network. A network, which may also be referred to as a graph, comprises a set of nodes which are connected by edges. The nodes of the graph data structure 43 correspond to proteins, and the edges correspond to the interactions between the proteins. The degree of a node, which is defined as the number of edges incident on a node, varies depending on the amount of interactions a protein has. Nodes that are within a small number of edges from a given node, for example, within 3, 4, or 5 edges, are defined as local to a node. The local interactome or local neighbourhood of a protein of interest can be found by identifying the proteins corresponding to the nodes that are local to the node representing the protein of interest.

The random walk circuitry 26 performs a series of random walks on a graph based on the graph data structure 43. A random walk is the generation of a series of random discrete steps in a mathematical space. When performed on graph, the random walk consists of a succession of random steps of length 1 through the graph, traversing from node to node. When landing at a given node, there is an equal probability of traversing any of the edges that are connected to that node. Performing random walks on the graph allows for the local neighbourhood around the starting node to be surveyed in an unbiased manner.

For each of the n nodes corresponding to a respective protein, i random walks of length j are performed, to make n×i random walks in total. The resulting data from the random walks are recorded as raw count data 45. For each of the n starting proteins 44, the filtering circuitry 28 summarizes the raw count data 45 and then normalizes it to result in normalized count data 47. The filtering circuitry 28 then filters the normalized count data 47 based on the degree of the each of the n starting nodes, which results in filtered count data 48. For each of the n proteins 44, the set of proteins that remain in the filtered count data 48 can be considered to belong to the local neighbourhood of that protein.

At stage 150, the display circuitry 32 displays the protein-protein interaction data stored in the graph data structure 43 on the display screen 16. The nodes of the graph are coloured based on the filtered count data 48 so that the user can visualize the local neighbourhood of each of the n proteins 44. The nodes are further coloured so that the user can see which proteins belong to a local neighbourhood of at least one of the n proteins 44.

Turning to FIG. 3, the method will now be considered in further detail.

At stage 200, the user receives a list of n drugs 40 and information stored in drug target databases 41. In one embodiment, the n drugs 40 are selected based on the medications that a patient is being prescribed or could potentially be prescribed. In the present embodiment, the n drugs 40 comprise a selective serotonin reuptake inhibitor (SSRI), a beta blocker, and a calcium channel blocker (CCB). In another embodiment, at least one of the drugs is a drug undergoing a clinical trial.

The user identifies a list of n proteins 44 that are each respectively associated with one of the n drugs 40. In the present embodiment, the n proteins 44 are drug targets of the respective drugs. In the present embodiment, the n proteins 44 are Depression Serotonin receptor (HTR3A), Anxiety Beta adrenergic receptor (ADRB1), and Hypertension L-type calcium channel (RYR1). In other embodiments, at least one of the n proteins 44 may be associated with one of the n drugs 40 because its activity is modulated by that drug, rather than being a direct target of that drug.

The drug targets may be identified based on the information contained in drug target databases 41 or from drug manufacturers. In the present embodiment, the n proteins 44 are determined by a user. In other embodiments, the n proteins may be determined automatically by the graph circuitry 24 based on the list of n drugs 40 and the drug target databases 41. The drug target databases 41 may be stored on the memory 37 or any suitable data store.

At stage 210, protein-protein interaction data 42 is received by the graph circuitry 24 from the memory 37 or any suitable data store. The protein-protein interaction data 42 comprises data representative of a set of proteins (or the set of genes that code for those proteins) and a representation of the interactions between the respective proteins. The protein-protein interaction data may comprise a subset of the known proteins in an organism, or it may include all known proteins in an organism. In the case of humans, the protein-protein interaction data can include all known human protein coding genes (˜20000).

The graph circuitry 24 converts the protein-protein interaction data 42 into a graph data structure 43 if the protein-protein interaction data 42 is not already in such a format. The graph data structure 43 is a simplified data structure suitable for representing a graph. Examples of a graph data structure include an edge list and an adjacency matrix. An edge list defines the start and end nodes of each of the edges in a graph, and can be represented as a two-column matrix. An adjacency matrix comprises rows and columns which are both labelled by the nodes, and a 1 or 0 entered in each of the cells according to whether an edge connecting the respective nodes is present or not. In other embodiments, any suitable graph data structure may be used.

In the present embodiment, the protein-protein interaction data 42 is received by the graph circuitry 24 in a format that is not a graph data structure 43, and it is converted by the graph circuitry 24 into a graph data structure 43. In other embodiments, the graph circuitry 24 may receive the protein-protein interaction data directly in the form of a graph data structure 43.

In the present embodiment, the graph data structure 43 corresponds to an unweighted graph, i.e. each of the edges of the graph has a weight of 1. In other embodiments, if the protein-protein interaction data comprises weighted interactions, such as data obtained from the STRING database, the graph circuitry 24 may first apply a threshold to the protein-protein interaction data to achieve unweighted protein-protein interaction data. The unweighted protein-protein interaction data is then converted to a graph data structure 43.

As an alternative, it is envisaged that in some embodiments, the weighted protein-protein interaction data is incorporated directly into the graph data structure 43 such that the graph data structure 43 comprises weighted edges.

At the end of stage 210, the graph circuitry 24 outputs the graph data structure 43.

At stage 220, the random walk circuitry 26 receives the graph data structure 43. The random walk circuitry also receives the list of n proteins 44. Each of the n proteins are used as a starting node with which to begin at least one random walk through a graph based on the graph data structure 43.

The random walk circuitry 26 performs i random walks for each of the n starting nodes, resulting in n×i random walks being performed in total, where i is an integer greater than or equal to 1. Each of the walks have a length j, where j is an integer greater than 1. Preferably, the length j of each of the random walks should be sufficient for capturing the local neighbourhood around a starting node n to allow for effective visualisation. This can be, for example, a length j of 4 or 5 steps. Longer walks can capture more distant interactions originating from a node.

It is envisaged that any suitable random walk algorithm may be used to perform the random walks on the graph based on the graph data structure 43. If the graph data structure 43 is weighted, a weighted random walk algorithm may be used whereby the probability of a transition between respective nodes is determined by the weight of the edge connecting those nodes.

The random walk circuitry 26 keeps a tally of the nodes visited over the i random walks for each of the n starting nodes, which is recorded as raw count data 45. At the end of stage 220, the random walk circuitry 26 outputs the raw count data 45.

At stage 230, the filtering circuitry 28 receives the raw count data 45. The filtering circuitry 28 summarises the raw count data 45 to result in summarised count data 46. The summarised count data 46 comprises, for each of the n starting nodes, a list of the proteins corresponding to the nodes visited over the i random walks, and an associated count value corresponding to the number of times a node associated with a respective protein is visited. The summarised count data 46 is stored as a single table or list indexed for each of the n starting nodes, or as a set of n tables or lists for each of the n starting nodes.

The count values in the summarised count data 46 are then normalised by i, which is the number of random walks performed for each of the n starting nodes. This results in normalised count data 47.

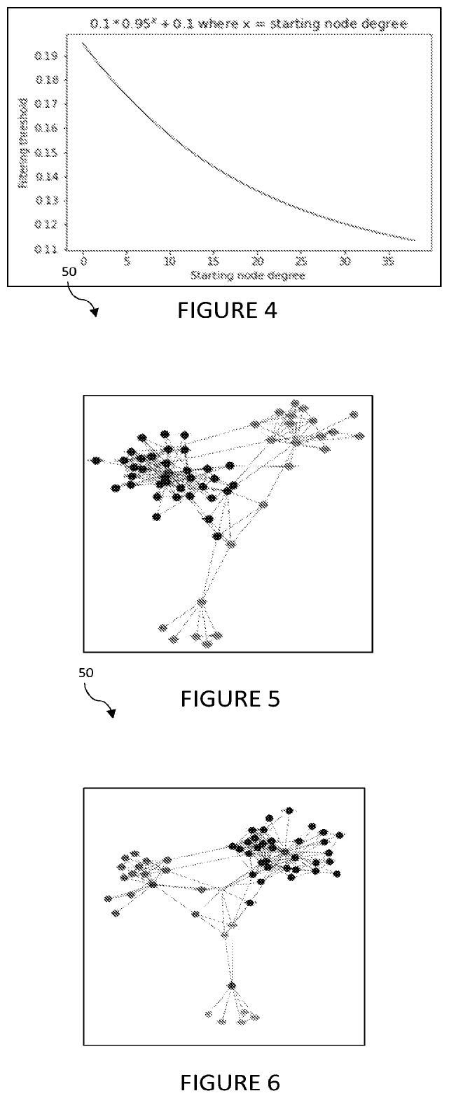

The filtering circuitry 28 then applies a threshold for each of the n sets of proteins in the normalised count data 47 to filter out proteins with low normalised count values. A threshold is chosen that is a function of the degree of the starting node. The degree of the starting node is obtained from the graph data structure 43. As illustrated in FIG. 4, the threshold, T, is chosen according to the function:

T=0.1×0.95^{x}+0.1, where x is the starting node degree.

Accordingly, the higher the starting degree of the node, the lower the threshold. Adjusting the threshold in this way takes into account the topology of the graph representing the protein-protein interaction data. A random walk starting on a node with a high degree tends to result in a many nodes being visited fewer times. A random walk starting on nodes with a low degree tends to result in fewer nodes being visited a greater number of times. The above function to calculate the threshold T adjusts the threshold in accordance with the degree of the nodes, so that the higher the starting node degree, the lower the threshold.

If each of the n starting nodes have a different degree, a different filtering threshold will be applied to each of the n sets of proteins.

At the end of stage 230, the filtering circuitry 28 outputs the filtered count data 48. The remaining proteins in the filtered count data 48 for each of the n starting nodes 44 may be referred to as the local neighbourhood or local interactome of that protein/associated drug.

At stage 240, the interaction score circuitry 30 receives the filtered count data 48.

The interaction score circuitry 30 calculates an interaction score 48 for each of the unique proteins in the filtered count data 48. The interaction score for a protein is calculated by finding the average count value for that protein over the n drugs 40. The interaction score may also be referred to as a gene neighbourhood overlap. If a protein is not present in one of the n sets of proteins in the filtered count data, it will be considered to have a count value of 0 for that set. As a consequence, proteins that are present in all of the n sets of proteins of the filtered count data 48 will tend to have a high interaction score. Of course, it is also possible that a protein that is visited a disproportionately high amount of times in only one or few of the n sets of proteins will also have a high interaction score. This will occur when a protein is strongly related to one or more of the n drugs 40 but is weakly linked to the remaining drugs.

At the end of stage 240, the interaction score circuitry 30 outputs the interaction scores 49 for each of the proteins.

At stage 250, the display circuitry 32 receives the interaction scores 49, the filtered count data 48 and the graph data structure 43. The display circuitry 32 generates a displaying image 50 of the graph based on these inputs and provides an indication of which proteins are influenced by which of the n drugs 40.

In the present embodiment illustrated in FIG. 5, the display circuitry 32 highlights the nodes corresponding to the proteins associated with only one of the n drugs 40 based on the filtered count data 48. In other words, the display circuitry 32 shows the local neighbourhood for one of the n drugs 40. The starting node is indicated by colouring the node (shown as greyscale in FIG. 3) differently to the other nodes. In other embodiments, other methods may be used to indicate the starting node, such as the node having a different size or shape compared to the other nodes. The display circuitry 32 further provides a display interface to allow a user to select which one, or which combination of, the n sets of proteins from the filtered count data 48 they wish to be displayed. This allows the user to explore the local interactome for each of the n drugs 40.

In the present embodiment illustrated in FIG. 6, the display circuitry 32 displays all of the sets of proteins from the filtered count data 48 simultaneously. Each of the nodes belonging to a respective set of proteins is shaded with a different colour, and each of the starting nodes is shaded with the same colour. For nodes corresponding to proteins that are part of more than one set of proteins, the nodes are further coloured or modified, or a graphical feature is added, to indicate the degree of overlap of that protein between sets of proteins.

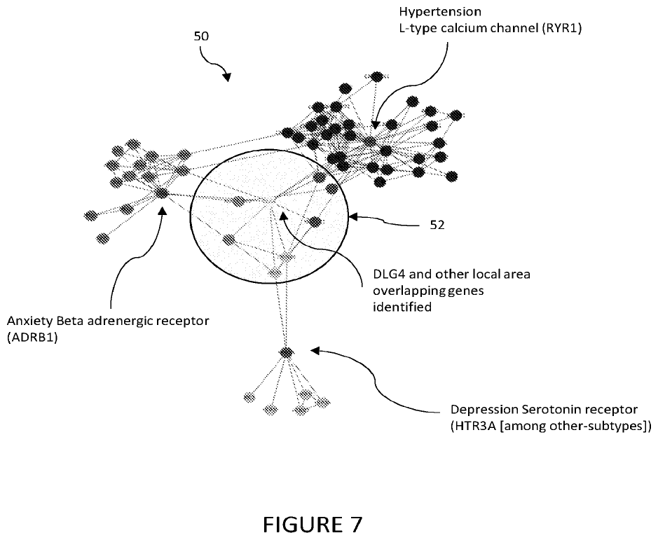

In the present embodiment illustrated in FIG. 7, the node representing protein DLG4 is present in each of the sets of proteins associated with the selective serotonin reuptake inhibitor (SSRI), a beta blocker, and a calcium channel blocker (CCB) respectively. The node representing protein DLG4 is shaded by a different colour to the other nodes in the graph. In alternative embodiments, a node corresponding to an overlapping protein is shaded by a combination of colours that indicate all of the drugs with which the protein corresponding to that node is associated. FIG. 7 further illustrates a circular region 52 which indicates the nodes that are present in more than one set of proteins. Although not shown, the interaction scores 49 may also be indicated on the image 50 by e.g. colouring a part of the node according to the interaction score or providing a mouse-over functionality to display the interaction score.

At stage 260, the analysis circuitry 34 receives the filtered count data 48 and graph data structure 43. Some or all of the proteins from the filtered count data 48 are then selected by the user or automatically by the analysis circuitry 34 for further analysis. The proteins selected for analysis may comprise a group of proteins that belong to the local interactomes of at least two or the n drugs 40.

The analysis circuitry 34 then performs gene set enrichment analysis (GSEA), pathway analysis or Uniprot analysis on all or some of the proteins in the filtered count data to explore possible biological mechanisms of side effects and adverse reactions when the n drugs 40 are administered to a patient. GSEA is a tool that associates biological pathways stored in the MSigDB database to a group of genes/proteins. Uniprot is a tool for associating functional information to a group of proteins. Based on the identified pathways, clinicians and researchers can determine possible side effects or adverse reactions when the n drugs are administered.

At stage 270, the analysis circuitry 34 then performs a further determining step based on the determined biological mechanisms, side effects or adverse reactions. Alternatively, the determining step may be performed by a clinician or researcher.

In one embodiment, a first drug is being administered in a clinical trial and the second drug is prescribed to a potential subject in the clinical trial. Stages 200, 210, 230, 240, 250 and 260 are evaluated based on the first drug and the second drug. At stage 270, the analysis circuitry determines, based on the biological mechanisms, side effects or adverse reactions, if the subject should be excluded from the clinical trial and/or a classification of the subject into a subgroup of the clinical trial participants. In another embodiment, a first drug and second drug are part of a treatment plan for a patient. Stages 200, 210, 230, 240, 250 and 260 are evaluated based on the first drug and the second drug. At stage 270, the analysis circuitry 34 determines if, based on the biological mechanisms, side effects or adverse reactions if one of the first or second drug should be removed from the treatment plan and/or if the dose of the first or second drug should be modified. In another embodiment, a first drug is a new drug candidate and a second drug is a commonly prescribed drug. Stages 200, 210, 230, 240, 250 and 260 are evaluated based on the first drug and the second drug. At stage 270, the analysis circuitry 34 determines, based on the biological mechanisms, side effects or adverse reactions, if the drug candidate should proceed to the drug development stage.

By performing a random walk on a PPI network, the local neighbourhood of a protein may be explored in an unbiased manner. By filtering the visited nodes, the topology of the graph is taken into account. This may offer improvement over a naïve method of exploring the graph, which would simply involve a number of hops from a drug target and does not take into account the density of the neighbourhood. Comparing the local neighbourhoods in the absence of a filter is less meaningful because it does not account for the density of neighbourhoods. The methods described above allows efficient discovery and display of local interactome, regardless of neighbourhood density. The output of the method is a list of genes/proteins that represent possible hubs of threshold polypharmacy interactions. This list can be mined for biological/clinical meanings.

The methods described above can be used by clinicians to examine patients responding poorly to a medication or to examine individuals with suspected adverse drug reactions (ADRs). Clinicians can input a patient-specific list of drug protein targets in challenging patient cases to obtain a visual representation of the effect of those drugs at the level of proteins. The visual representation can also be used in a teaching context to allow students to visualize polypharmacy networks with students.

The methods described above may also be a useful tool for a drug research and development process. For example, the methods may be used by researchers who wish to consider the effects of new drugs on patient biology, including possible biological effects, drug-protein/gene interactions, and possible contra-indications in polypharmacy patients. This information can be used to help decide if a potential new drug should pass a screening. The methods may be used for patient selection in clinical trial designs. If strong adverse effects are predicted, then the patient may be excluded from the clinical trial.

In other embodiments, different apparatus may be used to perform different processes, or parts of processes, of those described above. For example, a first apparatus may be used to convert the protein-protein interaction data, a second apparatus may be used to perform the random walks, a third apparatus may be used to perform the filtering, and a fourth apparatus may be used to perform the scoring, a fifth apparatus may be used to display the graph data, and a sixth apparatus may be used to perform further analysis. Any suitable combination of apparatuses may be used.

Whilst particular circuitries have been described herein, in alternative embodiments functionality of one or more of these circuitries can be provided by a single processing resource or other component, or functionality provided by a single circuitry can be provided by two or more processing resources or other components in combination. Reference to a single circuitry encompasses multiple components providing the functionality of that circuitry, whether or not such components are remote from one another, and reference to multiple circuitries encompasses a single component providing the functionality of those circuitries.

Certain embodiments provide a medical information processing apparatus comprising processing circuitry configured to: receive knowledge relating to interactions of proteins/genes, estimate, based on the knowledge, a first range based on a first drug and a second range based on a second drug, and determine, based on the first range and the second range, a target protein/gene which is influenced by both of the first drug and the second drug.

The knowledge may be a knowledge graph expressing the interactions of proteins/genes by using nodes and edges.

The processing circuitry may be further configured to estimate, by determining a first and a second node corresponding to the first drug and second drug based on the knowledge graph, the first range and second range by performing a first search and second search from the first node and the second node.

Certain embodiments provide a method of displaying the local area of effect of drug targets in a gene or protein network, in which nodes affected by a plurality of medications are identified and coloured by the intensity of interactions and outputted as a list of nodes that represent genes/proteins that can be used to characterise potential biological mechanisms of polypharmacy.

The drug targets may be treated as starting nodes for random walks, which are used for exploring local neighbourhood.

A function of the starting node degree may be used for determining the threshold for filtering the counts of visited nodes, which are used to identify the local neighbourhood of a drug.

The intersection of the set of visited nodes (after filtering) generated from random walks with different starting nodes may be used to identify genes/proteins affected by multiple medications.

The counts of the visited nodes (after filtering) generated from random walks with different starting nodes may be averaged to generate an interaction score.

The colour of the intersecting nodes among multiple neighbourhoods may be scaled by the interaction score.

The list of genes/proteins identified as enriched in overlapping drug neighbourhoods may be used as input for downstream analysis (e.g. gene-set enrichment analysis, Uniprot analysis) to infer biological mechanisms, side effects or adverse drug reactions.

The method of identifying local neighbourhood and overlaps between neighbourhoods may not be limited to the biological domain, and can be extended to graphs from other disciplines/domains.

The method of analysis and visualisation of polypharmacy may be applicable to organisms other than Homo sapiens.

Whilst certain embodiments have been described, these embodiments have been presented by way of example only, and are not intended to limit the scope of the invention. Indeed the novel methods and systems described herein may be embodied in a variety of other forms. Furthermore, various omissions, substitutions and changes in the form of the methods and systems described herein may be made without departing from the spirit of the invention. The accompanying claims and their equivalents are intended to cover such forms and modifications as would fall within the scope of the invention.

1. A medical information processing apparatus comprising processing circuitry configured to:
receive protein-protein interaction data;
estimate, using the protein-protein interaction data, a first set of proteins based on the first drug and a second set of proteins based on the second drug; and
determine, based on the first set of proteins and the second set of proteins, if there is at least one protein which is influenced by both the first drug and the second drug.

2. The medical information processing apparatus of claim 1, wherein the protein-protein interaction data comprises a protein-protein interaction, PPI, network.

3. The medical information processing apparatus of claim 2, wherein the intersection of the first set of proteins and the second set of proteins is used to estimate if there is at least one protein influenced by the first drug and the second drug.

4. The medical information processing apparatus of claim 3,
wherein the first set of proteins comprises a plurality of proteins visited during at least one random walk through the PPI network originating at a protein associated with the first drug, and
wherein the second set of proteins comprises a plurality of proteins visited during at least one random walk through the PPI network originating at a protein associated with the second drug.

5. The medical information processing apparatus of claim 4, wherein the protein associated with the first drug is a target of the first drug, and wherein the protein associated with the second drug is a target of the second drug.

6. The medical information processing apparatus of claim 5,
wherein each of the plurality of proteins in the first set of proteins has an associated count value equal to the number of times the protein is visited during the at least one random walk for the first drug,
and wherein each of the plurality of proteins in the second set of proteins has an associated count value equal to the number of times the protein is visited during the at least one random walk for the second drug.

7. The medical information processing apparatus of claim 6, wherein the processing circuitry is further configured to, after estimating the first set of proteins and the second set of proteins, and before determining the at least one protein influenced by both the first drug and the second drug; and
normalise the count values of each of the plurality of proteins in the first set of proteins and second set of proteins based on the number of random walks for the first drug and the second drug respectively.

8. The medical information processing apparatus of claim 7, wherein the processing circuitry is further configured to, after normalizing the count values, and before determining the at least one protein influenced by both the first drug and the second drug:
filter, based on the normalized count values, the first set of proteins and the second set of proteins using a first threshold for the first set of the proteins and a second threshold for the second set of proteins.

9. The medical information processing apparatus of claim 8, wherein the first threshold and the second threshold are calculated based on the degree of the node corresponding to the protein associated with the first drug and the second drug respectively, wherein the degree of a node is equal to the number of edges incident on a node.

10. The medical information processing apparatus of claim 9, wherein the first threshold and the second threshold are determined using the formula 0.1*0.95^{x}+0.1, wherein x is the degree of the node corresponding to the protein associated with the first drug and the second drug respectively.

11. The medical information processing apparatus of claim 10, wherein the processing apparatus is further configured to:
calculate an interaction score for each of the plurality of proteins in the first set of proteins and the second set of proteins based on the average normalised count values for said protein in the first set of proteins and the second set of proteins.

12. The medical information processing apparatus of claim 11, wherein the processing circuitry is further configured to:
display a subgraph of the PPI network, the subgraph comprising the first set of the proteins and the second set of proteins; and
visually indicate if a protein of the subgraph belongs to the first set of proteins, the second set of proteins, or both the first set and the second set of proteins.

13. The medical information processing apparatus of claim 12, wherein the processing circuitry is further configured to:
visually indicate the interaction score of at least one protein of the subgraph.

14. The medical information processing apparatus of claim 13, wherein the interaction score is represented by a colour scale.

15. The medical information processing apparatus of claim 14, wherein the processing circuitry is further configured to:
analyse the at least one protein influenced by the first drug and second drug to infer at least one of biological mechanism, side effect or adverse reaction in relation to treatment with the first drug and the second drug.

16. The medical information processing apparatus of claim 15, wherein the biological mechanisms are inferred by performing gene-set enrichment analysis, pathway analysis or Uniprot analysis.

17. The medical information processing apparatus of claim 15,
wherein the first drug is being administered in a clinical trial and the second drug is prescribed to a potential subject in the clinical trial, and wherein the processing circuitry is further configured to:
determine, based on the at least one biological mechanism, side effect or adverse reaction, if the subject should be excluded from the clinical trial and/or classify the subject into a subgroup within the cohort of clinical trial participants.

18. The medical information processing apparatus of claim 15,
wherein the first drug and the second drug relate to a treatment plan for a patient, and wherein the processing apparatus is further configured to:
determine, based on the at least one biological mechanism, side effect or adverse reaction, if one of the first or second drug should be removed from the treatment plan and/or if the dose of the first or second drug should be modified.

19. The medical information processing apparatus of claim 14, wherein the first drug is a new drug candidate and the second drug is a commonly prescribed drug, and wherein the processing apparatus is further configured to:
determine, based on the at least one biological mechanism, side effect or adverse reaction, if the drug candidate should proceed to the drug development stage.

20. A method comprising:
receiving protein-protein interaction data;
estimating, using the protein-protein interaction data, a first set of proteins based on the first drug and a second set of proteins based on the second drug;
estimating, based on the first set of proteins and the second set of proteins, if there is at least one protein which is influenced by both the first drug and the second drug.
